(19) United States
(12) Patent Application Publication
(10) Pub. No.: US 2017/0165178 A1
(43) Pub. Date: Jun. 15, 2017

(54) PRODUCT AND METHOD FOR TEMPORARY SHAPING OF KERATIN-CONTAINING FIBERS

(71) Applicant: Henkel AG & Co. KGaA, Duesseldorf (DE)

(72) Inventors: Thorsten Knappe, Schenefeld (DE); Pamela Kaftan, Hamburg (DE); Maria Catalina Bermudez Agudelo, Hamburg (DE); Tim Bethge, Hamburg (DE)

(73) Assignee: Henkel AG & Co. KGaA, Duesseldorf (DE)

(21) Appl. No.: 15/360,278

(22) Filed: Nov. 23, 2016

(30) Foreign Application Priority Data
Dec. 15, 2015 (DE) 10 2015 225 198.5

(51) Int. Cl. A61K 8/81; A61K 8/34; B65D 83/14; A61K 8/31; A61K 8/33; A61Q 5/06; A61K 8/04

(52) U.S. Cl. CPC A61K 8/8158; A61K 8/8147; A61Q 5/06; A61K 8/34; A61K 8/046; A61K 8/31; A61K 8/315; A61K 8/33; B65D 83/752; A61K 2800/594; A61K 2800/87

(57) ABSTRACT

Cosmetic products and methods for temporarily shaping keratinic fibers using the cosmetic products are provided herein. A cosmetic product for temporarily shaping keratinic fibers includes a cosmetic preparation. The cosmetic preparation contains at least one copolymer a1) that is constructed from at least the following monomer units: N-tert-octylacrylamide, acrylic acid, and tert.-butylaminoethyl methacrylate. The cosmetic preparation further contains at least one copolymer a2) that is constructed from at least the following monomer units: styrene, and at least one of acrylic acid or methacrylic acid. The proportion by weight of copolymers a1) and a2) with respect to the total weight of the cosmetic preparation is about 10 to about 30 wt %.

### CROSS-REFERENCE TO RELATED APPLICATION

This application claims priority to German Patent Application No. 10 2015 225 198.5, filed Dec. 15, 2015, which is incorporated herein by reference in its entirety.

### TECHNICAL FIELD

This disclosure relates to a cosmetic product for setting hair and for the temporary reshaping of keratinic fibers, particularly human hairs, the composition containing a combination of two specific copolymers in a high concentration.

### BACKGROUND

The temporary structuring of hairstyles for an extended period of time lasting up to several days generally requires the use of setting agents. Hair treatment products that serve to temporarily shape the hair therefore play an important role. Such products for temporary shaping usually contain synthetic polymers and/or waxes as the setting agent. Products for supporting the temporary reshaping of keratin-containing fibers can be manufactured as hair spray, hair wax, hair gel, or styling mousse, for example.

The most important characteristic of a product for temporarily shaping hair, hereinafter also called styling product, is that the greatest possible hold is given to the treated fibers in their newly modeled shape—i.e., a shape that is impressed upon the hair. This is also referred to as a strong hairstyle hold or high holding strength of the styling product. The hairstyle hold is determined substantially by the type and quantity of the setting agents used, although the other components of the styling product can also have an impact.

Besides a high holding strength, styling products must meet a whole series of other requirements. These can be roughly subdivided into characteristics on the hair, characteristics of the respective formulation, e.g., characteristics of sprayed aerosols, and characteristics pertaining to the handling of the styling product, with particular importance being placed on the characteristics on the hair. Particularly noteworthy are moisture resistance, low stickiness (tack), and a balanced conditioning effect. Moreover, a styling product should, to the greatest possible extent, be universally usable for all hair types and be gentle on hair and skin.

In order to meet the various requirements, a multitude of synthetic polymers have already been developed that are used as setting agents in styling products. These polymers can be subdivided into cationic, anionic, nonionic and amphoteric setting polymers.

Known amphoteric polymers that are used in hair-setting products are based on the monomer N-tert-octylacrylamide, acrylic acid, and tert.-butylaminoethyl methacrylate. Corresponding polymers and the use thereof in hair-styling gels are described, for example, in international application WO 2011/012464 A2.

Hair-setting products based on copolymers of styrene with (meth)acrylic acid and/or esters thereof are described in international patent application WO2012/168035 A1.

In principle, not every polymer and not every polymer blend is suitable for the manufacture of hair styling products. This is particularly true of hair sprays, in which the viscosity and therefore also the spraying behavior is influenced by the polymer and the quantity of polymer used, for example.

Furthermore, not every polymer and every polymer blend is suitable for the manufacture of highly concentrated hair styling products. Besides the problems with the viscosity and controllability of the products, as the polymer concentration increases, difficulties arise with respect to the solubility and storage stability of hair sprays in particular.

A hair spray with a high polymer content (compact hair spray) is described, for example, in international patent application WO 2005/012588 A2.

Even though suitable polymers and polymer combinations have been developed for use in styling concentrates in the area of temporary hair-shaping for quite some time, the results that have been achieved thus far continue to leave room for improvement, particularly with regard to the storage stability, applicability and holding strength of these products. In particular, styling products that are currently available can still be improved in the sense that a good combination of holding strength and long-lasting hold (high-humidity curl retention) is not always sufficiently ensured.

### BRIEF SUMMARY

Cosmetic products and methods for temporarily shaping keratinic fibers using the cosmetic products are provided herein. In one embodiment, a cosmetic product for temporarily shaping keratinic fibers includes a cosmetic preparation. The cosmetic preparation contains at least one copolymer a1) that is constructed from at least the following monomer units: N-tert-octylacrylamide, acrylic acid, and tert.-butylaminoethyl methacrylate. The cosmetic preparation further contains at least one copolymer a2) that is constructed from at least the following monomer units: styrene, and at least one of acrylic acid or methacrylic acid. The proportion by weight of copolymers a1) and a2) with respect to the total weight of the cosmetic preparation is about 10 to about 30 wt %.

In another embodiment, a method for temporarily shaping keratinic fibers includes the step of loading keratin-containing fibers with a cosmetic product. The cosmetic product contains at least one copolymer a1) that is constructed from at least the following monomer units: N-tert-octylacrylamide, acrylic acid, and tert.-butylaminoethyl methacrylate. The cosmetic product further contains at least one copolymer a2) that is constructed from at least the following monomer units: styrene, and at least one of acrylic acid or methacrylic acid. The proportion by weight of copolymers a1) and a2) with respect to the total weight of the cosmetic product is about 10 to about 30 wt %. The method further includes the step of temporarily fixing the keratin-containing fibers into shape.

### DETAILED DESCRIPTION

The following detailed description is merely exemplary in nature and is not intended to limit the invention or the application and uses of the invention. Furthermore, there is not intention to be bound by any theory presented in the preceding background or the following detained description.

It is contemplated herein to make additional suitable polymer combinations available that are characterized by good film-forming and/or setting characteristics and have a very high holding strength without having to sacrifice flexibility and good moisture resistance—particularly perspiration and water resistance. The polymer combinations were to also be suitable for the manufacture of concentrated cosmetic products having a high level of chemical and physical stability and be easy to apply.

This was achieved as contemplated herein through a combination of two specific, mutually different copolymers.

The following is contemplated herein:

1. A cosmetic product for temporarily shaping keratinic fibers, comprising

a) a cosmetic preparation containing
a1) at least one copolymer that is constructed from at least the following monomer units:
   N-tert-octylacrylamide,
   acrylic acid
   tert.-butylaminoethyl methacrylate;
a2) at least one copolymer that is constructed from at least the following monomer units:
   styrene
   acrylic acid and/or methacrylic acid,
      wherein the proportion by weight of copolymers a1) and a2) with respect to the total weight of the cosmetic preparation is about 10 to about 30 wt %.
      2. The cosmetic product according to point 1, wherein the proportion by weight of copolymers a1) and a2) with respect to the total weight of the cosmetic preparation is about 11 to about 25 wt % and particularly about 12 to about 20 wt %.
      3. The cosmetic product according to any one of the preceding points, wherein the at least one copolymer a1) consists, with respect to its total weight, of at least 90 wt %, preferably at least 95 wt % and particularly at least 97 wt % of the monomers N-tert-octylacrylamide, acrylic acid, and tert.-butylaminoethyl methacrylate.
      4. The cosmetic product according to any one of the preceding points, wherein the copolymer a1) consists of at least 90 wt %, preferably at least 95 wt % and particularly at least 97 wt % of the monomers N-tert-octylacrylamide, acrylic acid, tert.-butylaminoethyl methacrylate, methyl methacrylate, and hydroxypropyl methacrylate.
      5. The cosmetic product according to any one of the preceding points, wherein the copolymer a1) bears the INCI designation octylacrylamide/acrylates/butylaminoethyl methacrylate.
      6. The cosmetic product according to any one of the preceding points, wherein the preparation contains, with respect to its total weight, about 1.0 to about 15 wt %, preferably about 2.0 to about 14 wt % and particularly from about 4.0 to about 12 wt % copolymer a1).
      7. The cosmetic product according to any one of the preceding points, wherein the at least one copolymer a2) consists, with respect to its total weight, of at least 90 wt %, preferably at least 95 wt % and particularly at least 97 wt % of the monomers

styrene

acrylic acid and/or methacrylic acid.

8. The cosmetic product according to any one of the preceding points, wherein the at least one copolymer a2) consists, with respect to its total weight, of at least 90 wt %, preferably at least 95 wt % and particularly at least 97 wt % of the monomers

styrene

acrylic acid and/or methacrylic acid

acrylic acid ester and/or methacrylic acid ester

9. The cosmetic product according to any one of the preceding points, wherein the copolymer a2) bears the INCI designation styrene/acrylates copolymer.
10. The cosmetic product according to any one of the preceding points, wherein the preparation contains, with respect to its total weight, about 1.0 to about 15 wt %, preferably about 2.0 to about 14 wt % and particularly from about 4.0 to about 12 wt % copolymer a2).
11. The cosmetic product according to any one of the preceding points, wherein the weight ratio of copolymer a1) to copolymer a2) is from about 1:7 to about 7:1, preferably from about 1:5 to about 5:1 and particularly from about 1:3 to about 3:1.
12. The cosmetic product according to any one of the preceding points, wherein the preparation contains, with respect to its total weight, about 30 to about 90 wt %, preferably about 40 to about 85 wt % and particularly from about 50 to about 80 wt % ethanol.
13. The cosmetic product according to any one of the preceding points, wherein the preparation contains, with respect to its total weight, about 0.01 to about 30 wt %, and particularly about 5.0 to about 25 wt % water.
14. The cosmetic product according to any one of the preceding points, wherein the preparation consists, with respect to its total weight, of at least 70 wt %, preferably at least 80 wt % and particularly at least 90 wt % of copolymers a1) and a2), ethanol and water.
15. The cosmetic product according to any one of the preceding points, wherein the preparation a) has a viscosity (Brookfield DV2T viscometer; 20° C., spindle 2, 10 rpm) from about 10 to about 1000 mPas, preferably from about 15 to about 500 mPas and particularly from about 20 to about 200 mPas.
16. The cosmetic product according to any one of the preceding points, wherein the product further comprises

b) at least one propellant.

17. The cosmetic product according to any one of the preceding points, wherein the product further comprises
b) at least one propellant from the group propane, mixture of propane and butane, dimethyl ether and 1,1-difluoroethane.
   18. The cosmetic product according to any one of the preceding points, comprising, with respect to its total weight,

a) 30 to 60 wt % of the cosmetic preparation

b) 40 to 70 wt % propellant.

19. A comprehensive product, comprising

i) a cosmetic product according to any one of points 1 to 18

ii) a dispensing device with spray valve.

20. The cosmetic product according to point 19, wherein the spray valve has a maximum valve opening below 0.4 mm, preferably between about 0.22 and about 0.32 mm and particularly between about 0.25 and about 0.30 mm.
21. A use of a preparation or of a product according to any one of points 1 to 20 for temporarily shaping keratin-containing fibers, particularly human hair.
22. A method for temporarily shaping keratin-containing fibers, particularly human hair, in which the keratin-containing fibers are loaded with a cosmetic product according to any one of points 1 to 18 and temporarily fixed in their shape.
23. The method according to point 22, wherein the loading of the keratinic fibers is performed at a spray rate of the cosmetic preparation from about 18 to about 30 g/min, preferably from about 22 to about 28 g/min.

In the context of the present disclosure, it was surprisingly observed that, by combining two inherently known components that are already used in styling products, improved moisture resistance can be obtained in styling products. Other characteristics usually demanded of styling products, such as long-lasting hold, stiffness and low stickiness, are maintained. Such a good combination of characteristics could not be expected even with knowledge of the individual components and was surprising. It was observed through experimentation that the combination of the two components yielded a highly superadditive, i.e., synergistic effect with regard to the moisture resistance and holding strength.

As contemplated herein, the term “keratinic fibers” includes furs, wool and feathers, but particularly human hair.

The essential components of the cosmetic product as contemplated herein are the amphoteric copolymer a1) and the anionic acrylate copolymer a2), which is different from the copolymer a1).

A first essential component of cosmetic compositions as contemplated herein is the copolymer a1). With regard to the manufacturability, applicability and cosmetic effect of cosmetic products as contemplated herein, it has proven advantageous if the proportion by weight of the copolymer a1) with respect to the total weight of the cosmetic preparation a) is about 1.0 to about 15 wt %, preferably about 2.0 to about 14 wt % and particularly from about 4.0 to about 12 wt %.

The copolymer a1) is attributable to the monomers N-tert-octylacrylamide, acrylic acid, tert.-butylaminoethyl methacrylate and, optionally, other monomers.

Preferred copolymers a1) preferably consist of at least 90 wt %, preferably at least 95 wt % and particularly at least 97 wt % of the monomers N-tert-octylacrylamide, acrylic acid, and tert.-butylaminoethyl methacrylate. Preferably, the copolymers a1) are obtained using exclusively the monomers N-tert-octylacrylamide, acrylic acid and tert.-butylaminoethyl methacrylate.

Copolymers a1) from the monomers N-tert-octylacrylamide, acrylic acid, tert.-butylaminoethyl methacrylate, methyl methacrylate, and hydroxypropyl methacrylate are especially preferred. It is especially preferred particularly if the copolymer a1) consists of at least 90 wt %, preferably at least 95 wt % and particularly at least 97 wt % of the monomers N-tert-octylacrylamide, acrylic acid, tert.-butylaminoethyl methacrylate, methyl methacrylate, and hydroxypropyl methacrylate.

The previously described copolymers a1) are sold by National Starch, for example, under the name Amphomer® (INCI designation: octylacrylamide/acrylates/butylaminoethyl methacrylate copolymer; CAS number 70801-07-9).

The cosmetic preparations as contemplated herein contain an anionic acrylate copolymer a2) as the second essential component.

With regard to the manufacturability, applicability and cosmetic effect of cosmetic products as contemplated herein, it has proven advantageous if the proportion by weight of the copolymer a2) with respect to the total weight of the cosmetic preparation a) is about 1.0 to about 15 wt %, preferably about 2.0 to about 14 wt % and particularly from about 4.0 to about 12 wt %.

The copolymer a2) is attributable to the monomers i) styrene and ii) acrylic acid and/or methacrylic acid and, optionally, other monomers.

Preferred copolymers a2) preferably consist of at least 90 wt %, preferably at least 95 wt % and particularly at least 97 wt % of the monomers styrene and acrylic acid and/or methacrylic acid. Especially preferred copolymers a) were obtained exclusively from the monomers styrene and acrylic acid and/or methacrylic acid.

The cosmetic products according to another preferred embodiment are characterized in that the at least one copolymer a2) consists, with respect to its total weight, of at least 90 wt %, preferably at least 95 wt % and particularly at least 97 wt % of the monomers

styrene

acrylic acid and/or methacrylic acid

acrylic acid ester and/or methacrylic acid ester.

The previously described copolymers a2) are sold by Dow Chemicals, for example, under the name Acudyne® Shine (INCI designation: styrene/acrylates copolymer; CAS number 9010-92-8).

The copolymer a2) is preferably used in the cosmetic preparation in partially neutralized or neutralized form. At least one alkanolamine is preferably used for the neutralization. The alkanolamines that can be used as an alkalizing agent as contemplated herein are preferably selected from primary amines with a C₂-C₆ alkyl parent having at least one hydroxyl group. Especially preferred alkanolamines are selected from the group consisting of 2-aminoethane-1-ol (monoethanolamine), tris(2-hydroxyethyl)-amine (triethanolamine), 3-aminopropane-1-ol, 4-aminobutane-1-ol, 5-aminopentane-1-ol, 1-aminopropane-2-ol, 1-aminobutane-2-ol, 1-aminopentane-2-ol, 1-aminopentane-3-ol, 1-aminopentane-4-ol, 3-amino-2-methylpropane-1-ol, 1-amino-2-methylpropane-2-ol, 3-aminopropane-1,2-diol, 2-amino-2-methylpropane-1,3-diol. Alkanolamines that are very especially preferred as contemplated herein are selected from the group of 2-aminoethane-1-ol, 2-amino-2-methylpropane-1-ol and 2-amino-2-methyl-propane-1,3-diol. 2-amino-2-methylpropanol has proven to be an especially suitable neutralizing agent. Cosmetic products that are preferred as contemplated herein contain at least one alkanolamine, preferably 2-amino-2-methylpropanol. The 2-amino-2-methylpropanol is preferably used in the cosmetic preparations as contemplated herein in a quantity that does not exceed the quantity required for the neutralization of the copolymer a2). Preferably, the quantities of 2-amino-2-methylpropanol used in the cosmetic preparations as contemplated herein are 80 to 100%, especially preferably 90 to 100% and particularly 95 to 100% of the quantity required for the complete neutralization of the copolymer a2). In a preferred embodiment, the proportion by weight of the 2-amino-2-methylpropanol with respect to the total weight of the cosmetic preparation a) is about 0.2 to about 10 wt %, preferably about 0.5 to about 8.0 wt % and particularly about 1.0 to about 7.0 wt %.

The proportion by weight of copolymers a1) and a2) with respect to the total weight of the cosmetic preparation is about 10 to about 30 wt %. Preferably, the cosmetic product in which the proportion by weight of copolymers a1) and a2) with respect to the total weight of the cosmetic preparation is about 11 to about 25 wt % and particularly about 12 to about 20 wt %.

Besides the total proportion by weight of copolymers a1) and a2), the weight ratio of copolymers a1) and a2) in relation to one another has an influence on the moisture resistance, the holding strength, and the other application characteristics of cosmetic products as contemplated herein. Cosmetic products that are technically especially advantageous are characterized in that the weight ratio of copolymer a1) to copolymer a2) is from about 1:7 to about 7:1, preferably from about 1:5 to about 5:1 and particularly from about 1:3 to about 3:1.

Besides the previously described copolymers a1) and copolymers a2), the cosmetic preparations as contemplated herein can contain other active substances, adjuvants and care ingredients.

A first group of active substances that are preferably used are the film-forming polymers. These film-forming polymers are not identical to the copolymer a1) or copolymer a2) described previously. The proportion by weight of the film-forming polymer with respect to the total weight of the cosmetic preparation is about 0.1 to about 8.0 wt %, preferably about 0.5 to about 6.0 wt % and particularly about 1.0 to about 4.0 wt %.

Nonionic polymers are especially preferably used as film-forming polymers. Some examples of suitable nonionic polymers are:
Vinyl pyrrolidone/vinyl ester copolymers, such as those which are sold under the trade name Luviskol® (BASF), for example. Luviskol® VA 64 and Luviskol® VA 73, each vinyl pyrrolidone/acetate copolymers, are preferred nonionic polymers.
Cellulose ethers, such as hydroxypropyl cellulose, hydroxyethyl cellulose and methylhydroxypropyl cellulose, such as those which are sold under the trade names Culminal® and Benecel® (AQUALON), for example.
Shellac.
Polyvinyl pyrrolidones, such as those which are sold under the name Luviskol® (BASF), for example.
Siloxanes. These siloxanes can be both water-soluble and water-insoluble. Both volatile and nonvolatile siloxanes are suitable, with nonvolatile siloxanes being understood as being such compounds whose boiling point at atmospheric pressure lies above 200° C. Preferred siloxanes are polydialkyl siloxanes, such as polydimethyl siloxane, for example; polyalkylaryl siloxanes, such as polyphenylmethyl siloxane, for example; ethoxylated polydialkyl siloxanes, and polydialkyl siloxanes containing amine or hydroxy groups.
Glycosidically substituted silicones.

Due to their cosmetic effect in combination with the copolymers a1) and a2), film-forming polymers that are preferably used as contemplated herein are particularly the polyvinyl pyrrolidones (INCI designation: PVP) and the vinyl pyrrolidone/vinyl acetate copolymers (INCI designation VP/VA copolymers), with the proportion by weight of these polymers preferably being limited to quantities between about 1.0 and about 10 wt %. Especially preferred cosmetic preparations as contemplated herein are therefore characterized in that they also contain, with respect to their total weight, about 1.0 to about 10 wt % polyvinyl pyrrolidone and/or vinyl pyrrolidone/vinyl acetate copolymer, preferably polyvinyl pyrrolidone. Especially preferred cosmetic preparations have a proportion by weight of the polyvinyl pyrrolidone and/or vinyl pyrrolidone/vinyl acetate copolymer c) with respect to the total weight of the cosmetic preparation from about 2.0 to about 8.5 wt %, preferably from about 3.0 to about 7.0 wt %.

In summary, with the copolymers a1) and a2) as well as the film-forming polymer a3), cosmetic products that are especially preferred as contemplated herein contain three mutually different polymers.

Protein hydrolysates and/or derivatives thereof can be used as a care ingredient. Protein hydrolysates are product mixtures that are obtained through the acidic, basic or enzymatic decomposition of proteins. As contemplated herein, the term “protein hydrolysates” is understood as also referring to total hydrolysates and to individual amino acids and derivatives thereof, as well as to mixtures of different amino acids. The molecular weight of the protein hydrolysates that can be used as contemplated herein lies between about 75, the molecular weight of glycine, and about 200,000; preferably, the molecular weight is about 75 to about 50,000, and very especially preferably about 75 to about 20,000 Dalton.

Another group of care ingredients are the vitamins, provitamins, vitamin precursors and/or derivatives thereof. Those vitamins, provitamins and vitamin precursors are preferred which are usually associated with groups A, B, C, E, F and H.

Other care ingredients are glycerin, propylene glycol, panthenol, caffeine, nicotinamide, and sorbitol.

In addition to plant extract, mono- or oligosaccharides and/or lipids can also be used as a care ingredient.

The composition of some cosmetic preparations a) in which the proportion by weight of copolymers a1) and a2) with respect to the total weight of the cosmetic preparation is about 10 to about 30 wt %, preferably about 11 to about 25 wt % and particularly about 12 to about 20 wt %, can be found in the following table (information in wt % is with respect to the total weight of the cosmetic product unless indicated otherwise).

| | Formula 1 | Formula 2 | Formula 3 | Formula 4 | Formula 5 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Optional additives | ad 100 | ad 100 | ad 100 | ad 100 | ad 100 |
| | Formula 6 | Formula 7 | Formula 8 | Formula 9 | Formula 10 |
| Octylacrylamide/ | 1.0 to 15 | 1.5 to 14.5 | 2.0 to 14 | 3.0 to 13 | 4.0 to 12 |
| acrylates/ | | | | | |
| butylaminoethyl | | | | | |
| methacrylate | | | | | |
| copolymer | | | | | |
| (INCI) | | | | | |
| Copolymer a2) * | 1.0 to 15 | 1.5 to 14.5 | 2.0 to 14 | 3.0 to 13 | 4.0 to 12 |
| Optional additives | ad 100 | ad 100 | ad 100 | ad 100 | ad 100 |
| | Formula 11 | Formula 12 | Formula 13 | Formula 14 | Formula 15 |
| Copolymer a1) * | 1.0 to 15 | 1.5 to 14.5 | 2.0 to 14 | 3.0 to 13 | 4.0 to 12 |
| Styrene/acrylates | 1.0 to 15 | 1.5 to 14.5 | 2.0 to 14 | 3.0 to 13 | 4.0 to 12 |
| copolymer (INCI) | | | | | |
| Optional additives | ad 100 | ad 100 | ad 100 | ad 100 | ad 100 |
| | Formula 16 | Formula 17 | Formula 18 | Formula 19 | Formula 20 |
| Octylacrylamide/ | 1.0 to 15 | 1.5 to 14.5 | 2.0 to 14 | 3.0 to 13 | 4.0 to 12 |
| acrylates/ | | | | | |
| butylaminoethyl | | | | | |
| methacrylate | | | | | |
| copolymer | | | | | |
| (INCI) | | | | | |
| Styrene/acrylates | 1.0 to 15 | 1.5 to 14.5 | 2.0 to 14 | 3.0 to 13 | 4.0 to 12 |
| copolymer (INCI) | | | | | |
| Optional additives | ad 100 | ad 100 | ad 100 | ad 100 | ad 100 |

Preferred cosmetic products are based on an aqueous, aqueous/alcoholic or alcoholic carriers.

Preferred cosmetic products contain about 40 to about 98 wt %, preferably about 60 to about 95 wt % and particularly about 70 to about 92 wt % polar solvent, preferably polar solvent from the group of water, ethanol and isopropanol.

As mentioned previously, the lower alcohols with 1 to 4 carbon atoms that are usually used for cosmetic purposes, such as ethanol and isopropyl, for example, can be used as alcohols.

Besides these alcoholic solvents, water-soluble cosolvents can still also be used, particularly in combination with water. Examples of especially preferred cosolvents are glycerin and/or ethylene glycol and/or 1,2-propylene glycol, which are preferably used in a quantity from 0 to about 30 wt % with respect to the cosmetic preparation a).

Together with the copolymers a1) and a2) described above, the aqueous, aqueous/alcoholic or alcoholic carriers preferably constitute a substantial component of preparations a) as contemplated herein. Cosmetic preparations are especially preferred which, with respect to their total weight, consist of at least 70 wt %, preferably at least 80 wt % and particularly at least 90 wt % of copolymers a1) and a2), ethanol and/or water.

The composition of some technically advantageous cosmetic preparations a) with liquid carrier in which the proportion by weight of copolymers a1) and a2) with respect to the total weight of the cosmetic preparation is 10 to 30 wt %, preferably 11 to 25 wt % and particularly 12 to 20 wt %, can be found in the following table. (The data in wt % are in relation to the total weight of the cosmetic product unless indicated otherwise.)

| | Formula 21 | Formula 22 | Formula 23 | Formula 24 | Formula 25 |
|---|---|---|---|---|---|
| Copolymer a1)* | 1.0 to 15 | 1.5 to 14.5 | 2.0 to 14 | 3.0 to 13 | 4.0 to 12 |
| Copolymer a2)* | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |
| | Formula 26 | Formula 27 | Formula 28 | Formula 29 | Formula 30 |
| Octylacrylamide/ | 1.0 to 15 | 1.5 to 14.5 | 2.0 to 14 | 3.0 to 13 | 4.0 to 12 |
| acrylates/butylaminoethyl | | | | | |
| methacrylate copolymer | | | | | |
| (INCI) | | | | | |
| Copolymer a2)* | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |
| | Formula 31 | Formula 32 | Formula 33 | Formula 34 | Formula 35 |
| Copolymer a1)* | 1.0 to 15 | 1.5 to 14.5 | 2.0 to 14 | 3.0 to 13 | 4.0 to 12 |
| Styrene/acrylates | 1.0 to 15 | 1.5 to 14.5 | 2.0 to 14 | 3.0 to 13 | 4.0 to 12 |
| copolymer (INCI) | | | | | |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |
| | Formula 36 | Formula 37 | Formula 38 | Formula 39 | Formula 40 |
| Octylacrylamide/ | 1.0 to 15 | 1.5 to 14.5 | 2.0 to 14 | 3.0 to 13 | 4.0 to 12 |
| acrylates/butylaminoethyl | | | | | |
| methacrylate copolymer | | | | | |
| (INCI) | | | | | |
| Styrene/acrylates | 1.0 to 15 | 1.5 to 14.5 | 2.0 to 14 | 3.0 to 13 | 4.0 to 12 |
| copolymer (INCI) | | | | | |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

*according to claim 1

As will readily be understood, not only can the proportion by weight of the liquid carrier vary with respect to the total weight of the cosmetic preparation a), the weight ratio of aqueous to alcoholic carrier can also be changed.

Preferred cosmetic preparations contain, with respect to their total weight, about 0.01 to about 30 wt % and particularly about 5.0 to about 25 wt % water.

As mentioned previously, the lower alcohols with 1 to 4 carbon atoms that are usually used for cosmetic purposes, such as ethanol and isopropyl, for example, can be used as alcohols. Preferred cosmetic preparations contain, with respect to their total weight, about 30 to about 90 wt %, preferably about 40 to about 85 wt %, and particularly about 50 to about 80 wt % ethanol.

The composition of some technically advantageous cosmetic preparations a) with liquid carrier in which the proportion by weight of copolymers a1) and a2) with respect to the total weight of the cosmetic preparation is about 10 to about 30 wt %, preferably about 11 to about 25 wt % and particularly about 12 to about 20 wt %, can be found in the following table. (The data in wt % are in relation to the total weight of the cosmetic product unless indicated otherwise.)

| | Formula 41 | Formula 42 | Formula 43 | Formula 44 | Formula 45 |
|---|---|---|---|---|---|
| Copolymer a1)* | 1.0 to 15 | 1.5 to 14.5 | 2.0 to 14 | 3.0 to 13 | 4.0 to 12 |
| Copolymer a2)* | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | ad 100 | ad 100 | ad 100 | ad 100 | ad 100 |
| | Formula 46 | Formula 47 | Formula 48 | Formula 49 | Formula 50 |
| Octylacrylamide/ | 1.0 to 15 | 1.5 to 14.5 | 2.0 to 14 | 3.0 to 13 | 4.0 to 12 |
| acrylates/ | | | | | |
| butylaminoethyl | | | | | |
| methacrylate | | | | | |
| copolymer | | | | | |
| (INCI) | | | | | |
| Copolymer a2)* | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | ad 100 | ad 100 | ad 100 | ad 100 | ad 100 |
| | Formula 51 | Formula 52 | Formula 53 | Formula 54 | Formula 55 |
| Copolymer a1)* | 1.0 to 15 | 1.5 to 14.5 | 2.0 to 14 | 3.0 to 13 | 4.0 to 12 |
| Styrene/acrylates | 1.0 to 15 | 1.5 to 14.5 | 2.0 to 14 | 3.0 to 13 | 4.0 to 12 |
| copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | ad 100 | ad 100 | ad 100 | ad 100 | ad 100 |
| | Formula 56 | Formula 57 | Formula 58 | Formula 59 | Formula 60 |
| Octylacrylamide/ | 1.0 to 15 | 1.5 to 14.5 | 2.0 to 14 | 3.0 to 13 | 4.0 to 12 |
| acrylates/ | | | | | |
| butylaminoethyl | | | | | |
| methacrylate | | | | | |
| copolymer | | | | | |
| (INCI) | | | | | |
| Styrene/acrylates | 1.0 to 15 | 1.5 to 14.5 | 2.0 to 14 | 3.0 to 13 | 4.0 to 12 |
| copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | ad 100 | ad 100 | ad 100 | ad 100 | ad 100 |

*according to claim 1

The cosmetic preparation is preferably sprayed onto hair. This is done especially preferably using a propellant (aerosol spray). Preferred cosmetic products therefore comprise, in addition to the cosmetic preparation a), at least one propellant b).

Suitable propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, both individually and in combination. Hydrophilic propellant gases such as carbon dioxide, for example, can be used advantageously in terms of the present disclosure if the proportion of hydrophilic gases is selected so as to be low and lipophilic propellant gas (e.g., propane/butane) is present in excess. Propane, n-butane, iso-butane and mixtures of these propellant gases are especially preferred. Preferred cosmetic products are characterized in that the product further comprises at least one propellant b) from the group propane, mixture of propane and butane, dimethyl ether and 1,1-difluoroethane (INCI: hydrofluorocarbon 152a).

Preferred cosmetic products as contemplated herein contain, with respect to their total weight, about 30 to about 60 wt % of the cosmetic preparation a) and about 40 to about 70 wt % propellant b).

The rest of the composition of some preferred cosmetic products which, besides the cosmetic preparation a), also comprise a propellant b) and in which the proportion by weight of copolymers a1) and a2) with respect to the total weight of the cosmetic preparation is about 10 to about 30 wt %, preferably about 11 to about 25 wt % and particularly about 12 to about 20 wt %, can be found in table 1 below.

In table 1, the left column (“Formula x”) refers to a respective exemplary cosmetic preparation a) of formulas 1 to 60 listed in the tables disclosed further above. The other columns two through five (“Propellant”) each indicates the quantity of propellant combined with the corresponding cosmetic preparation. These data in “wt %” are in relation to the total weight of the cosmetic preparation a) of the respective “formula x” without propellant.

In the following table 1, the indication “50 to 200 wt %” corresponds to the addition of propellant to the cosmetic preparation a) in a quantity of 50 to 200 wt % of the weight of the cosmetic preparation a). In other words, in this cosmetic product, the cosmetic preparation a) and the propellant b) are present in a weight ratio from about 100:50 to about 100:200 or from about 2:1 to about 1:2.

The cosmetic products according to line 4, column 4 of table 1 below are therefore a mix of propellant-free cosmetic preparation a) according to formula 3 with a propane/butane mixture in a weight ratio of cosmetic preparation a) to propellant of about 100:50 to about 100:200. In other words, the entry on line 4, column 4 describes a cosmetic product for temporarily shaping keratinic fibers, comprising

a) a cosmetic preparation containing
a1) about 2.0 to about 14 wt % of at least one copolymer that is constructed from at least the following monomer units:
   N-tert-octylacrylamide,
   acrylic acid
   tert.-butylaminoethyl methacrylate;
a2) about 2.0 to about 14 wt % of at least one copolymer that is constructed from at least the following monomer units:
   styrene
   acrylic acid and/or methacrylic acid,

b) propellant from the group propane/butane mixtures,

wherein the proportion by weight of the copolymers a1) and a2) with respect to the total weight of the cosmetic preparation is about 10 to about 30 wt % and the weight ratio of cosmetic preparation a) to propellant b) is about 2:1 to about 1:2.

TABLE 1

| | Propellant [wt %] | | | |
|---|---|---|---|---|
| Formula 1 | 50 to 200 | 50 to 200 DFE* | 50 to 200 P/B** | 50 to 200 DME*** |
| Formula 2 | 50 to 200 | 50 to 200 DFE* | 50 to 200 P/B** | 50 to 200 DME*** |
| Formula 3 | 50 to 200 | 50 to 200 DFE* | 50 to 200 P/B** | 50 to 200 DME*** |
| Formula 4 | 50 to 200 | 50 to 200 DFE* | 50 to 200 P/B** | 50 to 200 DME*** |
| Formula 5 | 50 to 200 | 50 to 200 DFE* | 50 to 200 P/B** | 50 to 200 DME*** |
| Formula 6 | 50 to 200 | 50 to 200 DFE* | 50 to 200 P/B** | 50 to 200 DME*** |
| Formula 7 | 50 to 200 | 50 to 200 DFE* | 50 to 200 P/B** | 50 to 200 DME*** |
| Formula 8 | 50 to 200 | 50 to 200 DFE* | 50 to 200 P/B** | 50 to 200 DME*** |
| Formula 9 | 50 to 200 | 50 to 200 DFE* | 50 to 200 P/B** | 50 to 200 DME*** |
| Formula 10 | 50 to 200 | 50 to 200 DFE* | 50 to 200 P/B** | 50 to 200 DME*** |
| Formula 11 | 50 to 200 | 50 to 200 DFE* | 50 to 200 P/B** | 50 to 200 DME*** |
| Formula 12 | 50 to 200 | 50 to 200 DFE* | 50 to 200 P/B** | 50 to 200 DME*** |
| Formula 13 | 50 to 200 | 50 to 200 DFE* | 50 to 200 P/B** | 50 to 200 DME*** |
| Formula 14 | 50 to 200 | 50 to 200 DFE* | 50 to 200 P/B** | 50 to 200 DME*** |
| Formula 15 | 50 to 200 | 50 to 200 DFE* | 50 to 200 P/B** | 50 to 200 DME*** |
| Formula 16 | 50 to 200 | 50 to 200 DFE* | 50 to 200 P/B** | 50 to 200 DME*** |
| Formula 17 | 50 to 200 | 50 to 200 DFE* | 50 to 200 P/B** | 50 to 200 DME*** |
| Formula 18 | 50 to 200 | 50 to 200 DFE* | 50 to 200 P/B** | 50 to 200 DME*** |
| Formula 19 | 50 to 200 | 50 to 200 DFE* | 50 to 200 P/B** | 50 to 200 DME*** |
| Formula 20 | 50 to 200 | 50 to 200 DFE* | 50 to 200 P/B** | 50 to 200 DME*** |
| Formula 21 | 50 to 200 | 50 to 200 DFE* | 50 to 200 P/B** | 50 to 200 DME*** |
| Formula 22 | 50 to 200 | 50 to 200 DFE* | 50 to 200 P/B** | 50 to 200 DME*** |
| Formula 23 | 50 to 200 | 50 to 200 DFE* | 50 to 200 P/B** | 50 to 200 DME*** |
| Formula 24 | 50 to 200 | 50 to 200 DFE* | 50 to 200 P/B** | 50 to 200 DME*** |
| Formula 25 | 50 to 200 | 50 to 200 DFE* | 50 to 200 P/B** | 50 to 200 DME*** |
| Formula 26 | 50 to 200 | 50 to 200 DFE* | 50 to 200 P/B** | 50 to 200 DME*** |
| Formula 27 | 50 to 200 | 50 to 200 DFE* | 50 to 200 P/B** | 50 to 200 DME*** |
| Formula 28 | 50 to 200 | 50 to 200 DFE* | 50 to 200 P/B** | 50 to 200 DME*** |
| Formula 29 | 50 to 200 | 50 to 200 DFE* | 50 to 200 P/B** | 50 to 200 DME*** |
| Formula 30 | 50 to 200 | 50 to 200 DFE* | 50 to 200 P/B** | 50 to 200 DME*** |
| Formula 31 | 50 to 200 | 50 to 200 DFE* | 50 to 200 P/B** | 50 to 200 DME*** |
| Formula 32 | 50 to 200 | 50 to 200 DFE* | 50 to 200 P/B** | 50 to 200 DME*** |
| Formula 33 | 50 to 200 | 50 to 200 DFE* | 50 to 200 P/B** | 50 to 200 DME*** |
| Formula 34 | 50 to 200 | 50 to 200 DFE* | 50 to 200 P/B** | 50 to 200 DME*** |
| Formula 35 | 50 to 200 | 50 to 200 DFE* | 50 to 200 P/B** | 50 to 200 DME*** |
| Formula 36 | 50 to 200 | 50 to 200 DFE* | 50 to 200 P/B** | 50 to 200 DME*** |
| Formula 37 | 50 to 200 | 50 to 200 DFE* | 50 to 200 P/B** | 50 to 200 DME*** |
| Formula 38 | 50 to 200 | 50 to 200 DFE* | 50 to 200 P/B** | 50 to 200 DME*** |
| Formula 39 | 50 to 200 | 50 to 200 DFE* | 50 to 200 P/B** | 50 to 200 DME*** |
| Formula 40 | 50 to 200 | 50 to 200 DFE* | 50 to 200 P/B** | 50 to 200 DME*** |
| Formula 41 | 50 to 200 | 50 to 200 DFE* | 50 to 200 P/B** | 50 to 200 DME*** |
| Formula 42 | 50 to 200 | 50 to 200 DFE* | 50 to 200 P/B** | 50 to 200 DME*** |
| Formula 43 | 50 to 200 | 50 to 200 DFE* | 50 to 200 P/B** | 50 to 200 DME*** |
| Formula 44 | 50 to 200 | 50 to 200 DFE* | 50 to 200 P/B** | 50 to 200 DME*** |
| Formula 45 | 50 to 200 | 50 to 200 DFE* | 50 to 200 P/B** | 50 to 200 DME*** |
| Formula 46 | 50 to 200 | 50 to 200 DFE* | 50 to 200 P/B** | 50 to 200 DME*** |
| Formula 47 | 50 to 200 | 50 to 200 DFE* | 50 to 200 P/B** | 50 to 200 DME*** |
| Formula 48 | 50 to 200 | 50 to 200 DFE* | 50 to 200 P/B** | 50 to 200 DME*** |
| Formula 49 | 50 to 200 | 50 to 200 DFE* | 50 to 200 P/B** | 50 to 200 DME*** |
| Formula 50 | 50 to 200 | 50 to 200 DFE* | 50 to 200 P/B** | 50 to 200 DME*** |
| Formula 51 | 50 to 200 | 50 to 200 DFE* | 50 to 200 P/B** | 50 to 200 DME*** |
| Formula 52 | 50 to 200 | 50 to 200 DFE* | 50 to 200 P/B** | 50 to 200 DME*** |
| Formula 53 | 50 to 200 | 50 to 200 DFE* | 50 to 200 P/B** | 50 to 200 DME*** |
| Formula 54 | 50 to 200 | 50 to 200 DFE* | 50 to 200 P/B** | 50 to 200 DME*** |
| Formula 55 | 50 to 200 | 50 to 200 DFE* | 50 to 200 P/B** | 50 to 200 DME*** |
| Formula 56 | 50 to 200 | 50 to 200 DFE* | 50 to 200 P/B** | 50 to 200 DME*** |
| Formula 57 | 50 to 200 | 50 to 200 DFE* | 50 to 200 P/B** | 50 to 200 DME*** |
| Formula 58 | 50 to 200 | 50 to 200 DFE* | 50 to 200 P/B** | 50 to 200 DME*** |
| Formula 59 | 50 to 200 | 50 to 200 DFE* | 50 to 200 P/B** | 50 to 200 DME*** |
| Formula 60 | 50 to 200 | 50 to 200 DFE* | 50 to 200 P/B** | 50 to 200 DME*** |

*“DFE” corresponds to 1,1-difluoroethane
**“P/B” corresponds to a propane/butane mixture
***“DME” corresponds to dimethyl ether

Some vessels that are worthy of consideration as pressurized gas containers for aerosol applications are those made of metal (aluminum, tinplate, tin), protected or non-splitting plastic or of glass that is coated with plastic on the outside, with compressive and breaking strength, corrosion resistance, ease of fillability as well as aesthetic aspects, manageability, printability, etc., playing a role in the selection thereof. Special inner protective paints ensure corrosion resistance in relation to the cosmetic product a).

If the products as contemplated herein are to be sprayed onto the hair, these products are advantageously provided with a dispensing device and a spray valve. The resulting cosmetic products thus comprise a cosmetic product as contemplated herein as well as a dispensing device with spray valve. Spray valves are preferred which have a maximum valve opening below 0.4 mm, preferably between about 0.22 and about 0.32 mm and particularly between about 0.25 and about 0.30 mm. The valve opening surface area is preferably about 60×10⁻³ to about 100×10⁻³ mm².

In methods as contemplated herein, the spray rate is preferably from about 18 to about 30 g/min, particularly from about 22 to about 28 g/min.

The spray rate is determined not only by the pressure within the pressurized gas container and the valve opening, but also by the viscosity of the cosmetic preparation a). Preferred cosmetic products are characterized in that the preparation a) has a viscosity (Brookfield DV2T viscometer; 20° C., spindle 2, 10 rpm) from about 10 to about 1000 mPas, preferably from about 15 to about 500 mPas and particularly from about 20 to about 200 mPas.

In a preferred embodiment of the disclosure, the valve has a valve cone coated with a paint or a polymeric plastic A and such a flexible element with return characteristic that returns the valve to the closed position (=idle position of the valve) upon completion of actuation. Corresponding cosmetic products in which the aerosol dispensing device comprises a valve having a valve cone and/or a flexible element with return characteristic that is/are coated with a paint or a polymeric plastic A are preferred as contemplated herein.

In another preferred embodiment of the disclosure, the valve has a flexible element with return characteristic and/or a valve cone made of at least one plastic B, preferably an elastomeric plastic. Here as well, cosmetic products as contemplated herein in which the valve has a flexible element with return characteristic and/or a valve cone made of at least one plastic B, with preferred plastics B being elastomeric plastics. Especially preferred elastomeric plastics are selected from Buna, particularly Buna N, Buna 421, Buna 1602 and Buna KA 6712, neoprene, butyl and chlorobutyl.

In another preferred embodiment of the disclosure, the flexible element with return characteristic can be embodied as a spiral spring or helical compression spring. In another preferred embodiment of the disclosure, the flexible element with return characteristic can be integrally formed with the valve cone and have bendable legs.

As pointed out at the outset, the previously described cosmetic products are characterized by special cosmetic hair characteristics, particularly advantageous characteristics in the temporary hair-shaping. A second object of the present application is therefore the use of a product as contemplated herein for temporarily shaping keratin-containing fibers, particularly human hair.

A third object of the present application is a method for temporarily shaping keratin-containing fibers, particularly human hair, in which the keratin-containing fibers are loaded with a cosmetic product as contemplated herein and fixed temporarily in their shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A cosmetic product for temporarily shaping keratinic fibers, comprising:
a cosmetic preparation containing:
at least one copolymer a1) that is constructed from at least the following monomer units;
N-tert-octylacrylamide,
acrylic acid, and
tert.-butylaminoethyl methacrylate; and
at least one copolymer a2) that is constructed from at least the following monomer units;
styrene, and
at least one of acrylic acid or methacrylic acid;
wherein the proportion by weight of copolymers a1) and a2) with respect to the total weight of the cosmetic preparation is about 10 to about 30 wt %.

2. The cosmetic product as set forth in claim 1, wherein the proportion by weight of copolymers a1) and a2) with respect to the total weight of the cosmetic preparation is about 11 to about 25 wt %.

3. The cosmetic product as set forth in claim 1, wherein the preparation contains, with respect to its total weight, about 1.0 to about 15 wt % of copolymer a1).

4. The cosmetic product as set forth in claim 1, wherein the preparation contains, with respect to its total weight, about 1.0 to about 15 wt % of copolymer a2).

5. The cosmetic product as set forth in claim 1, wherein the preparation contains, with respect to its total weight, about 30 to about 90 wt % of ethanol.

6. The cosmetic product as set forth in claim 1, wherein the preparation contains, with respect to its total weight, about 0.01 to about 30 wt % of water.

7. The cosmetic product as set forth in claim 1, wherein the product further comprises at least one propellant.

8. A comprehensive product, comprising:
a cosmetic product as set forth in claim 1; and
dispensing device with spray valve.

9. (canceled)

10. (canceled)

11. The cosmetic product as set forth in claim 1, wherein the proportion by weight of copolymers a1) and a2) with respect to the total weight of the cosmetic preparation is about 12 to about 20 wt %.

12. The cosmetic product as set forth claim 1, wherein the preparation contains, with respect to its total weight, about 2.0 to about 14 wt % of copolymer a1).

13. The cosmetic product as set forth claim 1, wherein the preparation contains, with respect to its total weight, about 4.0 to about 12 wt % of copolymer a1).

14. The cosmetic product as set forth in claim 1, wherein the preparation contains, with respect to its total weight, about 2.0 to about 14 wt % of copolymer a2).

15. The cosmetic product as set forth in claim 1, wherein the preparation contains, with respect to its total weight, about 4.0 to about 12 wt % of copolymer a2).

16. The cosmetic product as set forth in claim 1, wherein the preparation contains, with respect to its total weight, about 40 to about 85 wt % of ethanol.

17. The cosmetic product as set forth in claim 1, wherein the preparation contains, with respect to its total weight, about 50 to about 85 wt % of ethanol.

18. The cosmetic product as set forth in claim 1, wherein the preparation contains, with respect to its total weight, about 5 to about 25 wt % of water.

19. The cosmetic product according to claim 1, wherein the at least one copolymer a1) consists, with respect to its total weight, of at least 90 wt % of the monomers N-tert-octylacrylamide, acrylic acid, and tert-butylaminoethyl methacrylate.

20. The cosmetic product according to claim 1, wherein the weight ratio of copolymer a1) to copolymer a2) is from 1:7 to 7:1.

21. A cosmetic product for temporarily shaping keratinic fibers, comprising:
a cosmetic preparation containing:
at least one copolymer a1) that is constructed from at least the following monomer units;
N-tert-octylacrylamide,
acrylic acid, and
tert.-butylaminoethyl methacrylate;
at least one copolymer a2) that is constructed from at least the following monomer units;
styrene, and
at least one of acrylic acid or methacrylic acid;
at least one propellant from the group propane, mixture of propane and butane, dimethyl ether and 1,1-difluoroethane;
ethanol; and
water;
wherein the proportion by weight of copolymers a1) and a2) with respect to the total weight of the cosmetic preparation is about 10 to about 30 wt %.

22. A method for temporarily shaping keratinic fibers, the method comprising the steps of:
loading keratin-containing fibers with a cosmetic product comprising:
at least one copolymer a1) that is constructed from at least the following monomer units;
N-tert-octylacrylamide,
acrylic acid, and
tert.-butylaminoethyl methacrylate; and
at least one copolymer a2) that is constructed from at least the following monomer units;
styrene, and
at least one of acrylic acid or methacrylic acid;
wherein the proportion by weight of copolymers a1) and a2) with respect to the total weight of the cosmetic product is about 10 to about 30 wt %; and
temporarily fixing the keratin-containing fibers into shape.
